(19) United States
(12) Patent Application Publication
(10) Pub. No.: US 2026/0027357 A1
(43) Pub. Date: Jan. 29, 2026

(54) SYSTEMS AND METHODS FOR DENTAL TREATMENT WITH EKF

(71) Applicant: T33 Dental, Inc., Cambridge, MA (US)

(72) Inventors: David Lundberg, Cambridge, MA (US); Daniel Lundberg, Cambridge, MA (US)

(21) Appl. No.: 19/284,259

(22) Filed: Jul. 29, 2025

Related U.S. Application Data
(60) Provisional application No. 63/676,700, filed on Jul. 29, 2024.

(51) Int. Cl. A61N 1/30; A61C 3/00; A61K 45/06; A61N 1/04; A61N 1/08

(52) U.S. Cl. CPC A61N 1/306; A61C 3/00; A61K 45/06; A61N 1/044; A61N 1/08

(57) ABSTRACT

This disclosure relates to systems and methods for the electrokinetic (EKF) delivery of therapeutic and cosmetic agents to oral tissues, including but not limited to enamel, dentin, cementum, gingiva, and mucosa. More specifically, it relates to modular, low-current dental treatment platforms configured to deliver active agents across or into oral tissues using flexible electrodes, voltage ramping logic, and optionally insulating or stabilizing structures.

### CROSS-REFERENCE TO RELATED APPLICATIONS

This application claims the benefit of priority to U.S. Provisional Patent Application No. 63/676,700 entitled “System and Method for Dental Treatment with EKF,” filed on Jul. 29, 2024, the contents of which are incorporated by reference herein in their entirety.

### FIELD OF THE INVENTION

The present invention relates to systems and methods for the electrokinetic (EKF) delivery of therapeutic and cosmetic agents to oral tissues, including but not limited to enamel, dentin, cementum, gingiva, and mucosa. More specifically, it relates to modular, low-current dental treatment platforms configured to deliver active agents across or into oral tissues using flexible electrodes, voltage ramping logic, and optionally insulating or stabilizing structures.

### BACKGROUND

Dental treatments for whitening, remineralization, desensitization, and antimicrobial purposes typically rely on passive diffusion, manual placement, or custom trays to deliver active compounds to target tissues. These methods often suffer from poor penetration, uneven distribution, or user discomfort. Furthermore, most existing systems lack dynamic control of treatment parameters and are not easily adaptable to varied anatomical configurations.

Electrical methods such as iontophoresis have been used to enhance drug delivery across biological tissues, including in dental contexts. However, these systems are often bulky, imprecise, or ill-suited for intraoral use due to discomfort, rigidity, or safety limitations. Additionally, conventional systems generally treat all teeth uniformly, without the ability to selectively deliver agents to specific regions.

Accordingly, there remains a need for a safe, modular, and patient-adaptable electrokinetic platform that enables precise, controllable delivery of various agents to specific intraoral surfaces-mineralized and non-mineralized alike—while incorporating comfort-enhancing and safety features suitable for clinical or at-home use.

### SUMMARY

In one aspect, the invention relates to a device for applying an electric potential to a treatment site. The device can include a power source adapted to provide energy for delivery to the treatment electrode; a treatment electrode adapted for placement proximate the treatment site; a reference electrode; and a control system. The control system can perform operations including: confirming that a complete circuit is established between the treatment electrode and the reference electrode; upon confirmation of the complete circuit, initiating a voltage ramp from an initial voltage to a target treatment voltage over a predetermined time interval; monitoring current delivered to the treatment electrode during the voltage ramp and a treatment period; and if the monitored current meets a predetermined condition, terminating voltage delivery.

In some embodiments, the treatment site can include a dental treatment site. The treatment electrode can be adapted to contact a conductive agent applied to the treatment site. The conductive agent can include a gel-formulated agent. The conductive agent can be a tooth whitening or shade-modifying agent, a remineralizing or desensitizing agent, and/or an antimicrobial or antibiotic agent. In some embodiments, the remineralizing or desensitizing agent includes fluoride salts, zinc salts, calcium phosphate compounds, and/or potassium nitrate. In some embodiments, the antimicrobial or antibiotic agent includes chlorhexidine, triclosan, and/or a tetracycline-class compound.

In some embodiments, the device can include an electrically insulating barrier adapted to be applied to a region adjacent the treatment site (e.g., gingival tissue). In some cases, the electrically insulating barrier is light-curable and adapted to conform to the region adjacent the treatment site. In some embodiments, confirming that the complete circuit is established includes applying a low-test voltage and detecting that a resulting current exceeds a test threshold value. In some embodiments, the device also includes a current-limiting element adapted to limit an amount of current delivered to the treatment electrode (e.g., a fixed resistor, a fuse, and/or a diode). In some cases, the device is adapted to interface only with components including an encoded connector, thereby restricting use to approved treatment conditions. The control system operations can also include generating an alert indicating a treatment status or fault condition.

In another aspect, the invention relates to an electrode for use in delivering a treatment composition to a treatment site. The electrode can include: a flexible and electrically conductive body, where the conductive body is customizable in size such that it can be physically adapted to conform to a particular treatment site; an electrical connection terminal adapted to couple the electrode to a power source, where the electrode is adapted to deliver electric current through the treatment composition to facilitate transport of active agents into the treatment site via at least one of electrokinetics and iontophoresis.

In some embodiments, the treatment site includes a dental treatment site. In some cases, the conductive body has a size sufficient to span across multiple teeth. The conductive body can include a laminated structure comprising a plastic backing layer, an adhesive layer, and a conductive metallic foil. The conductive body can be adapted to fit to the treatment site via at least one of structural perforations, expansion joints, and/or patterned slits. The electrode can be further adapted to be mechanically secured to a stabilizing structure during treatment. In some embodiments, the stabilizing structure is a check retractor. The electrode can be adapted to deliver electric current through the treatment composition to facilitate transport of active agents into the treatment site via at least one of electrophoresis and electroosmosis.

In another aspect, the invention relates to a retractor. The retractor can include a retractor element adapted to retract soft tissue from a treatment site; and at least one retention structure operably connected to the retractor element and adapted to removably secure a treatment component proximate the treatment site.

In some embodiments, the treatment site comprises a dental treatment site. The soft tissue can be a check tissue. The treatment component can be adapted to interact with a substance applied to the treatment site. The treatment component can at least partially enter the oral cavity. The treatment component can directly contact an intraoral tissue or substance. In some cases, the retention structure includes a clip, a hook, a loop, and/or a channel.

### BRIEF DESCRIPTION OF THE DRAWINGS

In the drawings, reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a system-level block diagram of an electrokinetic dental treatment system, according to various embodiments;

FIG. 2 is a schematic view of an electrokinetic dental treatment system applied to a patient, according to various embodiments;

FIG. 3A shows treatment of a single arch (e.g., upper anterior teeth), according to various embodiments;

FIG. 3B illustrates dual-arch treatment with simultaneous placement of electrodes across both the maxillary and mandibular arches, according to various embodiments;

FIG. 3C depicts treatment of non-contiguous teeth, according to various embodiments;

FIG. 4 is a top-down view of a flexible treatment electrode configured for electrokinetic delivery to dental surfaces, according to various embodiments;

FIG. 5A is a top-down view of the electrode in its compressed configuration, including slit or expansion features, according to various embodiments;

FIG. 5B illustrates the electrode of FIG. 5A in its extended form, where the expansion features open to accommodate curvature and increased span, according to various embodiments;

FIG. 5C depicts the electrode of FIGS. 5A-B conforming to a curved dental arch surface, with a contact interface ensuring consistent application of the treatment composition, according to various embodiments;

FIG. 6 is a side view of a flexible electrode placed in contact with a tooth surface via a layer of treatment composition, according to various embodiments;

FIG. 7A shows a cheek retractor placed on a patient, according to various embodiments;

FIG. 7B is a detailed view of a clip component of the retractor shown in FIG. 7A, according to various embodiments;

FIG. 8 illustrates a dual-arch intraoral stabilization using a multi-clip cheek retractor, according to various embodiments;

FIG. 9A shows a gingival barrier applied at the cervical margin of a treated tooth, according to various embodiments;

FIG. 9B depicts full encapsulation of the electrode according to various embodiments;

FIG. 10A illustrates two voltage application profiles: a step voltage curve that applies the full treatment voltage instantaneously, and a ramped voltage curve that gradually increases voltage over time;

FIG. 10B shows the resulting current through the patient under ramped voltage application, with a soft-start profile enabling a smooth rise to the desired treatment range;

FIG. 10C depicts a current-time profile where a brief disruption in circuit continuity-such as loss of contact between the electrode and the treatment site-temporarily interrupts current flow;

FIG. 10D illustrates a simulated short-circuit event occurring during treatment;

FIG. 10E shows an example system response when a short circuit or improper connection is present before treatment begins;

FIG. 11 is a flowchart of controller logic for pre-treatment current check, voltage ramping, and fault response, according to various embodiments;

FIG. 12 is a parameter chart showing exemplary ranges of certain operating parameters, according to various embodiments.

### DETAILED DESCRIPTION

Various embodiments of the present invention are directed to an improved treatment device. While this application will often describe the invention in connection with a dental treatment application, those skilled in the art will appreciate that the concepts described herein can be applied to other applications, including other medical or cosmetic applications. All descriptions herein, even if not expressly stated in the context of a particular description, should be interpreted as being according to only certain embodiments of the invention and examples and not limiting unless expressly stated in the claims.

While the embodiments described herein primarily focus on electrokinetic delivery to oral tissues such as enamel, dentin, cementum, gingiva, and mucosa, the systems and methods disclosed are not limited to dental applications. The electrokinetic principles, voltage control architectures, electrode configurations, and agent formulations described may be applied to other biological tissues, including but not limited to: skin, dermis, epidermis, subcutaneous tissue, cornea, sclera, conjunctiva, nasal or sinus mucosa, optic tissue, gastrointestinal lining, urogenital epithelium, or any other soft or mineralized tissue capable of interfacing with an externally applied electric field. These tissues may be targeted for therapeutic, diagnostic, cosmetic, or regenerative purposes. Accordingly, references to “dental” or “oral” treatment throughout this disclosure are intended as illustrative and non-limiting, and should not be construed as restricting the scope of the invention to intraoral or odonatological applications.

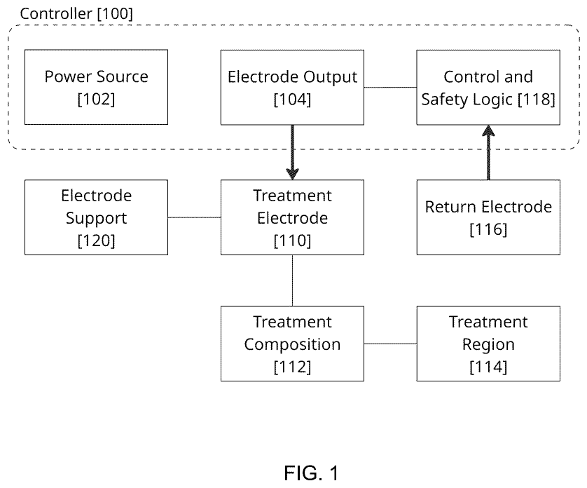

FIG. 1 is system-level block diagram of an electrokinetic dental treatment system comprising a controller 100 with an integrated power source 102 and electrode output port 104, operably connected to a treatment electrode 110 in contact with a treatment composition 112 applied to the treatment region 114. A return electrode 116 completes the electrical circuit. In some embodiments, the controller monitors circuit impedance 118 and regulates voltage application using ramping or current-limiting logic. A support structure 120, such as a check retractor, may optionally be used to stabilize the electrode or wire during treatment.

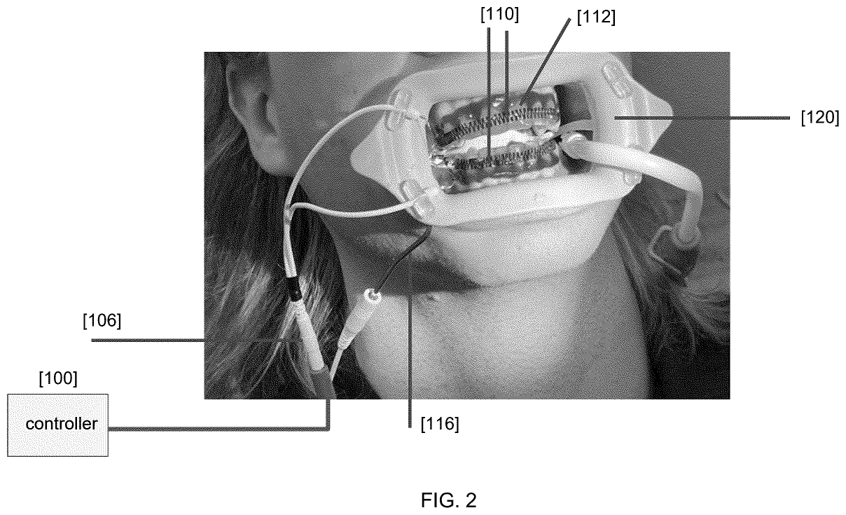

FIG. 2 is a schematic view of an electrokinetic dental treatment system applied to a patient, showing a flexible electrode 110 placed over the upper and lower arches and contacting a treatment composition 112 on the teeth 114. The electrode is stabilized by a check retractor 120, with the wire terminal 106 held in a retention clip. A return electrode 116 completes the electrical circuit, and a controller 100 supplies a ramped voltage to initiate treatment.

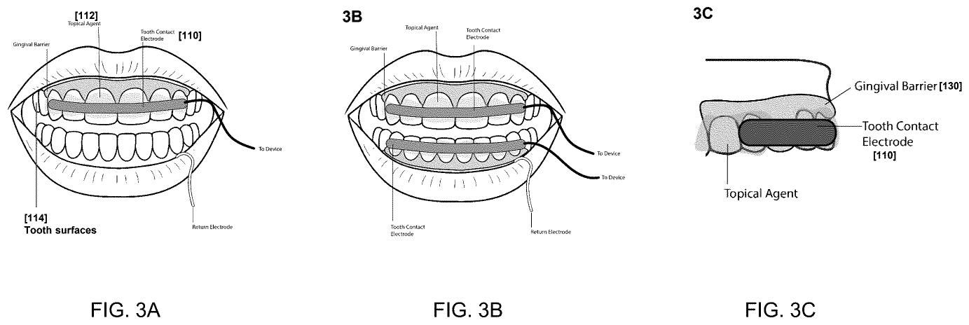

FIGS. 3A-C illustrate multiple example treatment configurations enabled by the electrokinetic dental treatment system. FIG. 3A shows treatment of a single arch (e.g., upper anterior teeth) with a flexible electrode 110 contacting a gel 112 on the tooth surfaces 114. FIG. 3B illustrates dual-arch treatment with simultaneous placement of electrodes across both the maxillary and mandibular arches. FIG. 3C depicts treatment of non-contiguous teeth, with the electrode 110 bridging gaps between treated teeth while intervening teeth are masked by an insulating barrier 130.

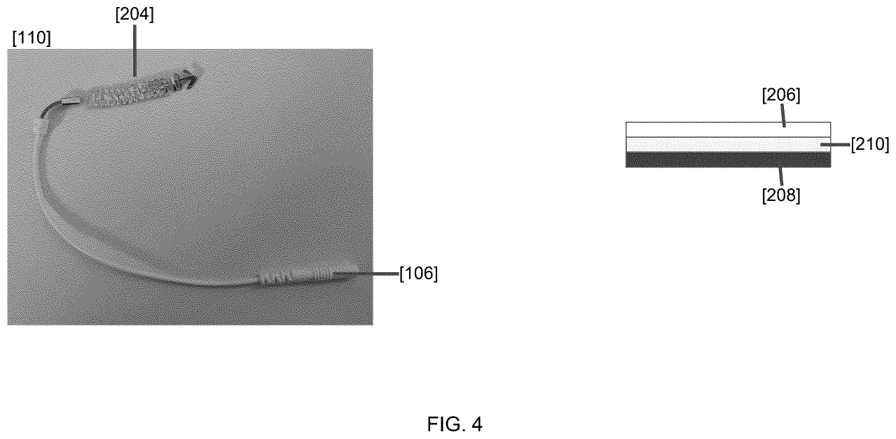

FIG. 4 is a top-down view of a flexible treatment electrode 110 configured for electrokinetic delivery to dental surfaces. The electrode includes a terminal region 106 for electrical connection to a controller. In some embodiments, the electrode includes a patterned expansion regions 204 that enable resizing or extension to accommodate different dental arch geometries. An optional cross-section illustrates example layered construction including a conductive layer 206, flexible substrate 208, and optional adhesive layer 210.

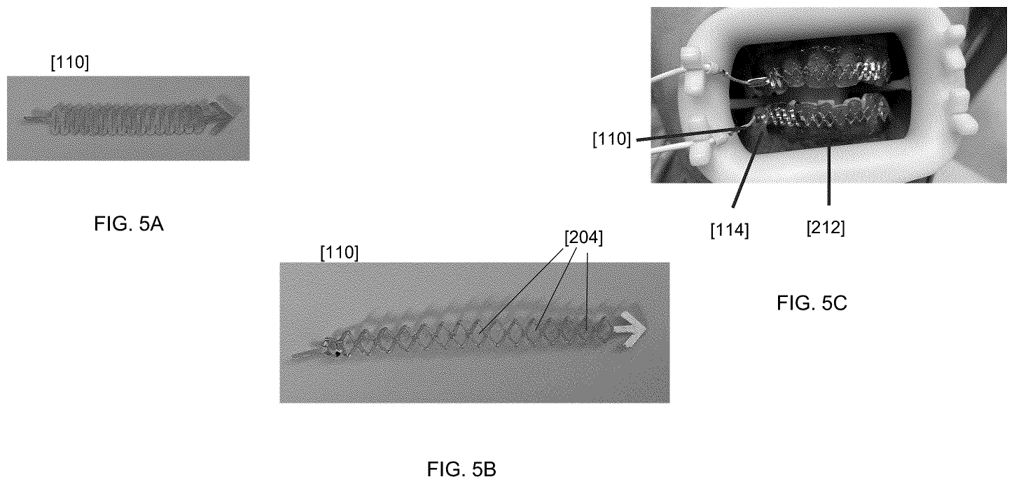

FIGS. 5A-C illustrate example electrode extensibility for arch conformity. FIG. 5A shows a top-down view of the electrode 110 in its compressed configuration, including slit or expansion features 204 designed to enable kirigami-style stretchability. FIG. 5B illustrates the same electrode 110 in its extended form, where the expansion features 204 open to accommodate curvature and increased span. FIG. 5C depicts the electrode 110 conforming to a curved dental arch surface 114, with a contact interface 212 ensuring consistent application of the treatment composition.

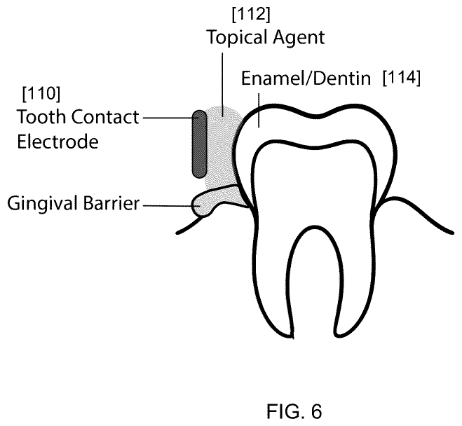

FIG. 6 is a side view of a flexible electrode 110 placed in contact with a tooth surface 114 via a layer of treatment composition 112. The electrode conforms to the curvature of the arch and rests on the gel without requiring compression, relying on surface tension or mechanical stabilization.

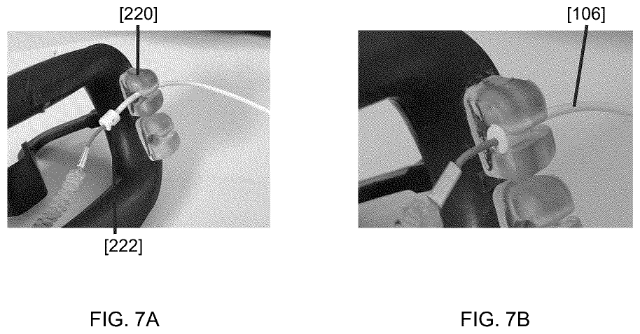

FIGS. 7A-B illustrate an example cheek retractor system for intraoral wire stabilization. FIG. 7A shows a cheek retractor 120 placed on a patient, supporting a treatment electrode wire 110 whose terminal end 106 is seated in a retention clip 220. FIG. 7B provides a detailed view of the clip 220, which includes an internal geometry that holds the wire terminal 106 in place using a flared tip or keyed engagement 222.

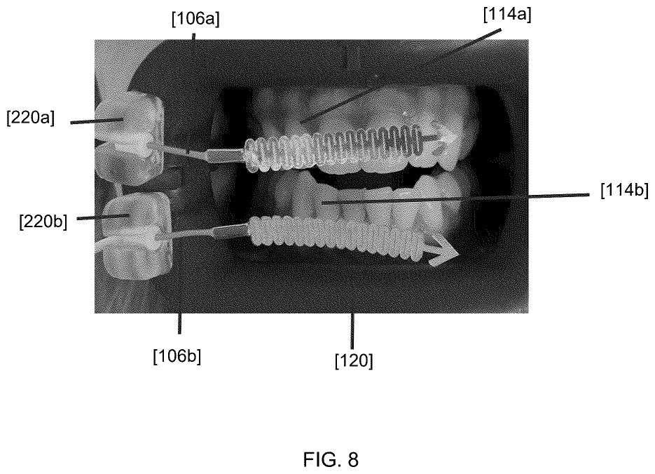

FIG. 8 is an example dual-arch intraoral stabilization using a multi-clip cheek retractor. A single retractor frame 120 includes two independent retention features 220a, 220b positioned to accept wire terminals 106a, 106b corresponding to upper and lower arch electrodes 110a, 110b. The retractor holds each wire in place during treatment, enabling full-mouth application without custom trays or active operator involvement. Upper and lower tooth arches 114a, 114b are shown for reference.

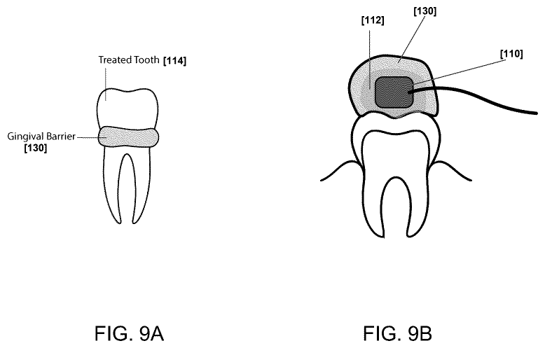

FIGS. 9A-B illustrate example gingival and selective insulation strategies for electrokinetic dental treatment. FIG. 9A shows a gingival barrier 130 applied at the cervical margin of a treated tooth 114 to insulate surrounding soft tissue and direct current through the enamel. FIG. 9B depicts full encapsulation of the electrode 110 and gel 112 under a barrier layer 130, providing precise current confinement and post-treatment retention.

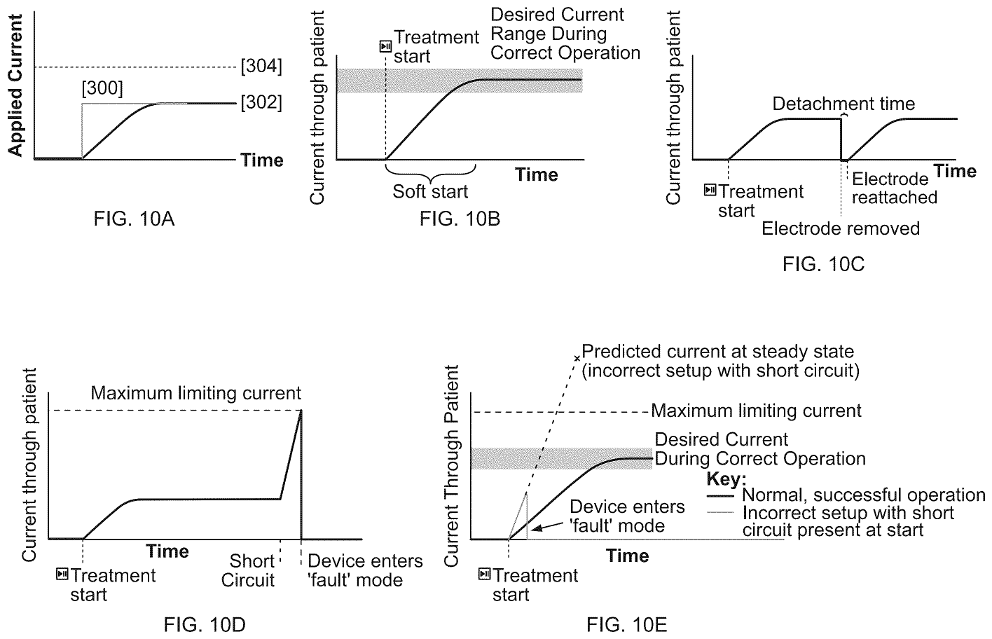

FIG. 10A illustrates two voltage application profiles: a step voltage curve 300 that applies the full treatment voltage instantaneously, and a ramped voltage curve 302 that gradually increases voltage over time. The perception threshold 304 indicates the voltage at which a patient may first feel tingling or stimulation, for example, between 5-7 V. Ramped voltage application reduces sudden current spikes and improves comfort, supporting real-time impedance monitoring and adaptive safety logic.

FIG. 10B shows the resulting current through the patient under ramped voltage application, with a soft-start profile enabling a smooth rise to the desired treatment range. This controlled ramp supports imperceptible delivery and enables continuous monitoring of tissue impedance.

FIG. 10C depicts a current-time profile where a brief disruption in circuit continuity—such as loss of contact between the electrode and the treatment site—temporarily interrupts current flow. Upon detecting the disconnection, the system recognizes that the circuit is incomplete, reduces the applied voltage to zero or near-zero levels, and subsequently re-initiates a soft-start ramp once continuity is restored. This prevents unintended high-voltage exposure or sudden current surges upon reconnection, thereby maintaining patient comfort and safety even in the case of momentary detachment or wire movement.

FIG. 10D illustrates a simulated short-circuit event occurring during treatment. After the initial ramp-up, the system detects a rapid and abnormal rise in current through the patient. This current exceeds both the expected treatment range and the system's defined maximum allowable current threshold. Because this level of current would not occur during normal electrokinetic operation-even at the target treatment voltage—the device interprets the event as a fault condition (e.g., a short circuit or misplacement of the electrode). The system responds by entering a fault mode and terminating voltage delivery to prevent discomfort or harm.

FIG. 10E shows the system's response when a short circuit or improper connection is present before treatment begins. During the initial soft-start phase, the system detects abnormally high current draw at low voltage, which would exceed the permissible treatment range if allowed to ramp to full voltage. Based on this early behavior, the system proactively shuts off before reaching levels that could cause discomfort or harm. This prediction can be based on a variety of real-time sensing methods, including but not limited to: measured impedance between electrodes, current response to applied low voltage, time-dependent current profiles, temperature rise, or other hardware- or software-based fault detection mechanisms. The control logic may implement thresholds, derivative analysis, or model-based predictions to determine when current behavior is incompatible with safe or effective electrokinetic delivery. This capability enables the system to preemptively identify risk conditions and avoid perceptible or unsafe electrical exposure.

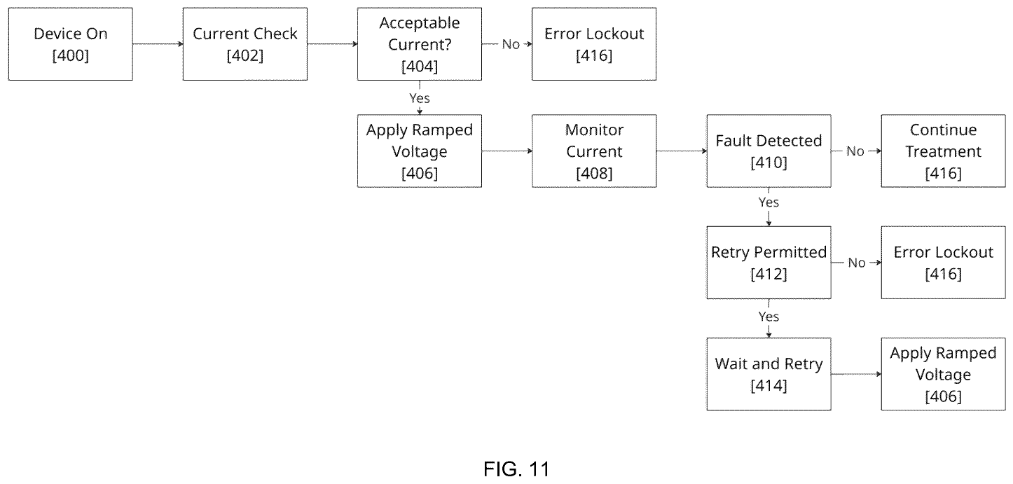

FIG. 11 is a flowchart of controller logic for pre-treatment current check, voltage ramping, and fault response. The process begins with a baseline impedance check 402. If the impedance is within acceptable limits 404, a ramped voltage 406 is applied while the system continuously monitors current 408. If a fault is detected 410, the controller may optionally attempt a retry 412-414; otherwise, the system halts treatment 416 for safety. This logic allows for safe, comfortable, and adaptive electrokinetic delivery.

### Electrokinetic Dental Treatment System Overview

Conventional dental treatments such as whitening, fluoride therapy, or desensitization rely on passive diffusion through enamel, which is slow, variable in efficacy, and difficult to control across multiple teeth. Light-based systems attempt to accelerate these processes, but they provide limited transport enhancement and may cause sensitivity or inconsistent results.

Electrokinetic flow (EKF) enables active, directional transport of charged and polar species across dental tissues using an applied electric field. While EKF has been validated in laboratory models, its adaptation to the clinical environment has faced challenges such as: achieving uniform treatment across multiple, variably sized teeth, managing patient comfort and perception during voltage application, preventing unintentional current paths through soft tissue, fitting the device to different patient anatomies, and/or avoiding complex or custom-fabricated trays.

The present invention discloses a modular electrokinetic treatment system designed for safe, effective, and scalable delivery of active agents into and across dental surfaces. In various embodiments, the system can include a controller 100 (see FIG. 1) that delivers a controlled voltage waveform to a treatment site or region 114; one or more soft or extensible electrodes 110 (see FIG. 2) configured to contact a gel-formulated agent applied to the teeth; an optional barrier layer 130 (see FIG. 9) to electrically isolate gingival or non-target tissue; a return electrode 116 (see FIG. 1) (e.g., lip hook or pad); and, in some embodiments, a check retractor 120 system (see FIG. 7) that holds and stabilizes the electrode and/or wire during treatment.

The system can allow for simultaneous treatment of one or multiple teeth on one or both arches, with minimal patient discomfort and a design adaptable to in-office or unsupervised contexts.

In some embodiments, the electrode is stabilized intraorally using a check retractor fitted with one or more retention structures 220 (see FIG. 7B) (e.g., clips, channels, or keyed receivers). These retractor-mounted clips hold the wire or electrode in position, reducing displacement due to wire tension, patient movement, or saliva flow. This retractor-electrode integration serves several roles: maintains electrical and mechanical stability of the electrode during treatment; avoids the need for custom mouthguards or molds; supports modular use of the system across patients enables rapid placement and repositioning by clinical staff; and/or can optionally house return electrodes 110 (see FIG. 2) (e.g., embedded lip hook contacts)

In some embodiments, the clip 220 (see FIG. 7B) component may be removable or disposable, featuring adhesive or mechanical attachment to other commercially available retractors. In others, the retractor and clip may be integrated as a single unit.

In some embodiments, various advantages of the invention can include: applying a constant or controlled voltage across an ionic or conductive gel 112 (see FIG. 2) ensures consistent electric field strength across treated surfaces. This enhances treatment consistency, even when tooth geometry or area or spacing varies between patients or application type. The system can be configured to treat single teeth, quadrants, or full arches. Electrodes may skip intervening restorations or gaps using bridging gel and modular placement. The integrated check retractor system maintains consistent electrode positioning across different patient anatomies and supports hands-free operation, improving workflow and user comfort. Electrode strips and retractor systems are designed for one-size-fits-most use, avoiding custom trays. In some cases, barrier 130 (see FIG. 9)-secured or retractor-supported treatment configurations allow for continued agent delivery even after the patient leaves the operatory.

The system supports the delivery of a wide range of agents, including but not limited to: (1) whitening agents, including but not limited to: hydrogen peroxide, carbamide peroxide, peracetic acid, phthalimidoperoxycaproic acid (PAP), calcium peroxide, sodium percarbonate, sodium perborate, urea peroxide, and other peroxide- or oxygen-releasing compounds; adjunctive accelerators or co-agents such as sodium bicarbonate, potassium nitrate, or polyphosphates; and formulations containing encapsulated, stabilized, or slow-release oxidizers designed to enhance tooth shade by oxidation of surface or subsurface chromogens. These agents may be delivered in liquid, gel, varnish, film, or emulsion formats, optionally in combination with thickening agents, stabilizers, pH buffers, or desensitizing additives, and are suitable for enhanced delivery using the electrokinetic systems described herein; (2) remineralization compounds, including but not limited to: fluoride salts (e.g., sodium fluoride, stannous fluoride, monofluorophosphate); calcium-based agents (e.g., calcium phosphate, calcium glycerophosphate, calcium lactate, calcium chloride, calcium silicate); phosphate sources (e.g., inorganic phosphate, potassium phosphate, sodium phosphate); amorphous calcium phosphate (ACP), functionalized calcium phosphate, and casein phosphopeptide-amorphous calcium phosphate (CPP-ACP) complexes; nano-hydroxyapatite, fluorapatite, and other enamel-mimetic nanomaterials; bioactive glass (e.g., 45S5 Bioglass®) and other ion-releasing glass compositions; zinc compounds (e.g., zinc oxide, zinc phosphate) with remineralizing or adjunctive antimicrobial activity; and other agents or formulations intended to restore enamel mineral content, increase surface hardness, reduce lesion depth, or occlude dentinal tubules; (3) desensitizers, including but not limited to: potassium salts such as potassium nitrate, potassium chloride, or potassium oxalate; strontium-based compounds (e.g., strontium chloride, strontium acetate); calcium-containing agents that promote tubule occlusion (e.g., calcium phosphate, calcium carbonate, nano-hydroxyapatite); oxalate-based formulations (e.g., ferric oxalate, potassium oxalate); protein-precipitating or nerve-calming agents such as glutaraldehyde or arginine bicarbonate; and other materials or complexes intended to reduce fluid flow within dentinal tubules, block neural response, or otherwise mitigate thermal or mechanical hypersensitivity. These agents may be incorporated into gels, varnishes, rinses, or other topical carriers and may be delivered more effectively via the electrokinetic systems described herein; (4) antimicrobials or antibiotics, including but not limited to: chlorhexidine (e.g., gluconate or acetate salt), triclosan, sodium hypochlorite, povidone-iodine, hydrogen peroxide, ozone-releasing agents, and essential oil components (e.g., thymol, eucalyptol, menthol, methyl salicylate); as well as antibiotics such as tetracycline, doxycycline, minocycline, metronidazole, ciprofloxacin, amoxicillin, clindamycin, erythromycin, and other locally or systemically active agents commonly used in endodontic or periodontal therapy. These materials may function to reduce bacterial load, disrupt biofilms, or suppress specific pathogenic organisms within carious lesions, gingival pockets, or restoration margins. Agents may be delivered alone or in combination with anti-inflammatory, desensitizing, or remineralizing compounds, and may be formulated as liquids, gels, pastes, varnishes, or slow-release systems for enhanced electrokinetic transport into mineralized or soft oral tissues; (5) anti-inflammatory agents, biologics, or peptides, including but not limited to: non-steroidal anti-inflammatory drugs (NSAIDs) such as ibuprofen or diclofenac; corticosteroids such as dexamethasone or triamcinolone; cytokine modulators or inhibitors (e.g., IL-1β, IL-6, TNF-α antagonists); antimicrobial peptides (e.g., LL-37, defensins, histatins); signaling peptides and growth factors such as TGF-β, BMPs, PDGF, FGF, or VEGF; and synthetic or recombinant proteins intended to modulate immune response, promote wound healing, or enhance tissue regeneration. These agents may be used to manage inflammation, accelerate healing, or influence cellular activity in periodontal, endodontic, or mucosal applications, and may be delivered as solutions, gels, hydrogels, or encapsulated systems using the electrokinetic delivery platforms described herein; (6) diagnostic tracers or sampling reagents, including but not limited to: fluorescent dyes (e.g., fluorescein, rhodamine, indocyanine green), radiopaque compounds (e.g., iodine-based agents, barium sulfate), photoacoustic or near-infrared contrast agents, pH-sensitive or redox-sensitive dyes, and ionic or molecular indicators that signal the presence of decay, demineralization, enzymatic activity, inflammation, or specific biomarkers. These reagents may be applied topically or integrated into treatment gels to support real-time visualization, pre- or post-treatment assessment, or quantitative monitoring of agent penetration or biological response. In some embodiments, the system may also be configured to facilitate electrokinetically assisted sampling of ionic species, biomarkers, or microbial content for diagnostic purposes, with delivery and collection occurring through the same or separate treatment interfaces; and/or (7) polymers, resins, or sealants, including but not limited to: dental adhesives, bonding agents, and primers; etchants such as phosphoric acid or maleic acid solutions; light-curable resins and hydrogels (e.g., based on BIS-GMA, UDMA, TEGDMA, HEMA, or PEG-based systems); flowable composites; glass ionomer cements and resin-modified ionomers; and moisture-tolerant or self-etching adhesives. These materials may be used to promote adhesion, occlude dentinal tubules, protect treated surfaces, or serve as carriers for bioactive agents. In certain embodiments, such materials may be applied before or after electrokinetic treatment to seal the delivery site, enhance bonding to restorative materials, or preserve therapeutic effects. Some polymerizable or dual-cure formulations may also function as barrier layers to prevent soft tissue exposure to electrical current or treatment compounds.

In various embodiments, these agents may be delivered into or through any combination of mineralized or non-mineralized oral tissues, including but not limited to enamel, dentin, cementum, gingiva, and mucosa. Treatment targets may include not only pristine enamel but also early-stage demineralization (e.g., white spot lesions), incipient caries, arrested or active lesions, and areas with structural compromise extending into the dentin or toward the pulp chamber. The system is likewise suitable for cosmetic, preventive, diagnostic, or therapeutic applications across a spectrum of oral conditions.

Additional therapeutic targets include early-stage decay prevention, such as remineralization or antimicrobial infiltration at sites vulnerable to demineralization or bacterial colonization.

In various embodiments, these agents may be delivered into or through any combination of mineralized or non-mineralized oral tissues, including but not limited to enamel, dentin, cementum, gingiva, and mucosa. Treatment targets may include not only pristine enamel but also early-stage demineralization (e.g., white spot lesions), incipient caries, arrested or active lesions, and areas with structural compromise extending into the dentin or toward the pulp chamber. The system is likewise suitable for cosmetic, preventive, diagnostic, or therapeutic applications across a spectrum of oral conditions.

In some embodiments, agent delivery may occur through electrokinetic forces such as electroosmosis or electrophoresis. In others, delivery may be enhanced by electrochemical interactions—including, without limitation, localized pH shifts, redox reactions, or catalytic activation—that facilitate agent transformation, targeting, or deeper tissue penetration.

As used herein, electrokinetic delivery refers to any form of electrically driven transport of chemical species into or through a target material or biological tissue. This includes, without limitation, agent movement arising from electroosmosis (bulk fluid flow induced by electric fields acting on charged boundary layers), electrophoresis (motion of charged molecules or particles in response to an electric field), and iontophoresis (current-assisted translocation of ionic agents across biological barriers). Collectively, these phenomena enable directional and controllable transport of active substances, including but not limited to drugs, cosmetic agents, and diagnostic tracers, into a variety of target media—including biological tissues such as enamel, skin, mucosa, or other mineralized or soft surfaces.

Unless explicitly stated otherwise, references to electrokinetic flow (EKF) or electrokinetic treatment are intended to encompass these field-driven transport mechanisms, individually or in combination, without limitation to any specific target tissue or anatomical region. While the present system may also generate secondary electrochemical effects—such as localized pH modulation, redox-driven activation, or electrophysical disruption of barriers—these effects are not required for electrokinetic delivery as defined herein, but may optionally be employed to enhance agent activity, transformation, or efficacy in certain embodiments.

In various embodiments, the platform can include: (1) electrodes: conformable strips that adhere via surface tension, or mechanical support, or an adhesive, optionally extensible via kirigami patterns (see below); (2) power supply and controller: a voltage source 102 (see FIG. 1) (e.g., battery with boost converter) featuring soft-start profiles, current limiting, and fault detection (see below); (3) barrier materials: polymerizable or flowable gels that isolate soft tissues, prevent leakage, or immobilize treatment zones (see below); (4) check retractor system: mechanically retains electrode wires or terminals, optionally includes keyed or indexed clips 220 (see FIG. 7B) to ensure proper connection and support (see below).

Example use cases of the system can include, but are not limited to: tooth whitening, enamel remineralization, sensitivity reduction, antimicrobial or anti-inflammatory treatment, adhesion priming, diagnostic sampling or agent deposition, and/or delivery to non-adjacent teeth or through restorations with isolation.

### Electrode System for Electrokinetic Dental Treatments

Electrokinetic transport requires a conductive pathway between a voltage source and the treatment site, typically across a gel or liquid formulation in contact with the target surface. Conventional dental electrode 110 (see FIG. 2), such as handheld probes or rigid metal components, are ill-suited for clinical use in multi-tooth treatment. They require manual placement, offer limited coverage, and are often incompatible with modern treatment workflows.

A clinically suitable electrode system should, in some cases: (1) deliver electric current effectively through the applied formulation, (2) conform to complex oral geometries, (3) enable treatment of one or multiple teeth with minimal operator input, and (4) maintain electrical and mechanical stability throughout the treatment period. In some embodiments, the electrode should be adaptable to different patient anatomies, reusable or single-use, and compatible with optional accessories such as check retractor 120 (see FIG. 7).

The disclosed electrode system comprises a flexible, electrically conductive body configured to contact a formulation applied to a dental surface. The electrode includes a terminal region 106 (see FIG. 4) connectable to an external power source and may be shaped or modified to conform to the desired treatment region. The electrode may be used to treat a single tooth, a partial arch, or a full arch, and may be positioned on the upper or lower dentition.

The electrode body is configured to remain in contact with the treatment site without requiring active compression. In some embodiments, this is achieved by surface tension with the applied gel 112 (see FIG. 2). In others, the electrode is stabilized mechanically using a retention mechanism associated with a check retractor system, as described in this application.

In certain embodiments, the electrode is pre-packaged with a treatment formulation, such as an agent-loaded hydrogel, enabling direct placement and activation without separate application of the gel. These integrated systems may simplify setup, improve consistency, and support single-use clinical workflows.

In other embodiments, a single connector or terminal may branch into multiple electrically active segments, enabling simultaneous treatment of spatially separated sites (e.g., two teeth on opposite sides of the arch). Electrodes may be shaped or modified to address different surfaces (e.g., facial, occlusal, or interproximal) or anatomical depths (e.g., enamel vs. dentin).

In various embodiments, the electrode body may be constructed from any electrically conductive material 206 (See FIG. 4) that is flexible and biocompatible. Suitable materials include, but are not limited to: carbon-filled polyethylene, electrically conductive polymers, conductive fabrics or threads, laminated films comprising a plastic substrate, adhesive interface, and conductive foil, metallized or plated meshes, metallic films such as aluminum foil or gold foil or thin stainless steel, composite structures containing reinforcing and conductive layers.

The material may be selected for compatibility with oxidative or acidic agents, resistance to degradation over the course of treatment, and case of handling by clinical staff. In some embodiments, the electrode comprises a composite structure optimized for both conductivity and mechanical stability. This composite structure may include a flexible backing material 208, potentially joined to the electrically conductive layer by an adhesive 210 or other sufficient method of attachment, such as thermal lamination, co-extrusion, mechanical bonding, or solvent-based film fusion, provided the resulting electrode maintains structural integrity and layer cohesion under normal clinical use.

The electrode may include stretchable, elastomeric regions or patterned adhesive layers to facilitate secure contact with curved surfaces. In certain embodiments, conductive adhesives or layered hydrogel structures may be used to enhance adherence or simplify placement.

In some embodiments, the electrode is a composite structure in which the electrically conductive layer does not itself provide primary mechanical support, but is instead coupled with one or more non-conductive or inert support layers—such as polymer films, adhesive backings, or structural laminates—that confer strength, flexibility, or shape stability. For example, a commercially implemented configuration may comprise a thin aluminum foil laminated to a polyvinylidene fluoride (PVDF) carrier with an intermediate adhesive layer. However, other combinations of mechanically supportive and electrically active layers are likewise contemplated, and the present disclosure is not limited to any particular material pairing or architecture.

To accommodate variability in patient dentition and clinical use cases, the electrode may be resized or reshaped. In some embodiments, the electrode is manufactured in an elongated or oversized format and is trimmed, folded, torn, or otherwise resized by the user to fit the treatment site. In other embodiments, the electrode incorporates mechanical cut patterns that enable in-plane extension or curvature when tension is applied. This may include kirigami-style slits, perforations, or patterned expansion joints 204 (see FIG. 5).

In various embodiments, the electrode may be used: as a single continuous piece extending across multiple teeth; as multiple discrete segments configured for selective placement; as a modular system of interconnectable pieces; and/or in a shortened configuration for treatment of a single tooth. The electrode may be positioned buccally, labially, interproximally, or occlusally, depending on the application.

In some embodiments, the electrode is constructed from materials and geometries that allow it to rest in stable contact with a gel, emulsion, or treatment composition 112 (see FIG. 2) via surface tension or light adhesion forces. The electrode may be sufficiently soft, flexible, and low in weight such that it does not require compression or mechanical force to remain in place. Conversely, it may avoid excessive stiffness or rigidity that would otherwise cause it to lift, dislodge, or fall from the treatment site under its own weight or due to minor anatomical irregularities.

In some implementations, the electrode incorporates kirigami-style cuts or slits that enable both mechanical compliance and electrical continuity. These structures may open under tension to increase length or curvature while maintaining a conductive path.

In certain embodiments, both the treatment and return electrodes may be integrated into a single intraoral substrate, such as a flexible tray, patch, or mouthpiece. This substrate may include embedded conductive paths, optional return electrodes, or elastic supports configured to conform to the dental arch.

In some embodiments, the electrode assembly may be supported or applied using a modular applicator, tray, or handle structure. These applicators may allow manual placement, alignment, or replacement of the electrode and may be configured for reuse or disposability.

In certain embodiments, the electrode is configured to not only extend during placement, but to include a controlled mechanical failure region—such as a frangible line, tear-away notch, or weakened cut pattern—that is designed to break or separate upon application of a defined pulling force. This breakage occurs in a predictable location, leaving behind an optimally sized and fully functional electrode that conforms to the desired treatment area. Such designs may include pre-weakened or perforated zones that serve as mechanical detents, allowing the clinician or user to confidently pull and extend the electrode until a clean release occurs, ensuring proper sizing without risk of under-extension or accidental damage. This approach provides a robust, user-friendly method for intraoral adjustment, accommodating patient variability while maintaining structural and electrical integrity.

In various embodiments, the electrode is configured to be connected to a voltage source via an electrical wire 106 (see FIG. 4) or conductive terminal. In some embodiments, the electrode includes a defined terminal region designed to receive a mating wire or clip. This terminal may include one or more of the following features: a flared or keyed geometry for mechanical engagement; a stop or butt end to prevent slippage during treatment; a clip 220 (see FIG. 7B), slot, or channel associated with a check retractor system for strain relief. In some embodiments, the electrode-wire connection is secured to a fixed structure (e.g., a retractor clip), preventing tension from displacing the electrode during patient movement or repositioning.

In some embodiments, the electrode is stabilized intraorally using a check retractor fitted with one or more attachment structures, as described in this application. This stabilizes the wire and electrode during use, allowing for hands-free treatment. The electrode may be suspended in contact with the treatment site, held in place by surface tension with the applied gel, or indirectly affixed via the check retractor.

In other embodiments, the electrode is enclosed partially or entirely within a cured barrier layer 130 (see FIG. 9) to maintain electrical contact and prevent contamination or leakage, as described in this application.

In various embodiments, the electrode is configured to deliver a direct current or time-varying voltage waveform from the controller 100 (see FIG. 1) to the applied treatment composition. The electrical potential causes migration of active species through the enamel or other targeted tissue via electrokinetic mechanisms including electrophoresis and electroosmosis.

The electrode system may be used in conjunction with: formulations containing whitening agents, remineralization agents, antimicrobials, desensitizers, or other therapeutics (see below); a controller capable of voltage ramping, current sensing, and fault response (see below); electrical return electrodes 116 (see FIG. 1) placed externally or intraorally; insulating barriers configured to direct current through the desired tissue region (see below). In some embodiments, the electrode system is capable of operating in both passive and active modes, with the controller adjusting voltage output based on current feedback or impedance monitoring.

Example configurations can include: a kirigami-cut aluminum-foil/plastic composite film electrode spanning the upper anterior 8 teeth; a single-tooth treatment strip trimmed to size and secured with retractor-mounted clips 220 (see FIG. 7B); dual-arch electrode strips configured to treat both maxillary and mandibular teeth simultaneously; and/or flexible adhesive-backed electrode conforming to the occlusal surfaces of posterior teeth.

In various embodiments, the system may include one or more of the following variations: electrodes configured for disposable or reusable use; electrodes with integrated sensors for impedance or temperature; preloaded electrode strips containing embedded or coated treatment agents; and/or electrodes with hydrophobic or non-conductive regions to limit current flow to selected areas. The disclosed electrode system supports flexible and extensible configurations for a wide range of clinical and non-clinical applications. It is compatible with the cheek retractor system described and the modular treatment platform described in this application.

### Check Retractor System for Electrode Stabilization

In dental treatments involving application of topical agents to teeth, it is common practice to retract the lips and cheeks using a check retractor 120 (see FIG. 7). This exposes the treatment area, maintains a dry field, and improves visibility and access. Certain whitening systems incorporate retractor-mounted lights or attachments, but existing retractors do not provide structural support for electrode systems or intraoral wiring.

When performing electrokinetic (EKF) delivery, electrode placement and wire 106 (see FIG. 4) routing are critical. If left unsupported, the electrode wire may exert force on the electrode strip, disrupting its position, displacing the treatment gel, or causing unintended electrical contact with soft tissue. This is particularly problematic in procedures where precision, comfort, and electrical isolation are essential.

There is a need for a check retractor system that can physically retain and support intraoral electrode 110 (see FIG. 2) and wires, ensuring stable treatment delivery without reliance on custom trays or operator holding.

The disclosed check retractor system includes one or more retention structures or mechanisms configured to support a treatment component—such as a flexible electrode or wire—during intraoral treatment. These retention features may be integrated into the retractor itself, affixed via adhesive or mechanical means, or configured as modular, removable clip 220 (see FIG. 7B). In some embodiments, the retention structure is designed to hold a wire or electrode terminal in a fixed position, allowing the active electrode surface to rest on the patient's teeth with minimal tension or movement. In other embodiments, the retractor system includes custom geometry that accommodates known electrode form factors and prevents rotation, slipping, or misplacement.

The retractor system may be used for single-arch or dual-arch treatments, and may optionally support return electrode 116 (see FIG. 1) as well.

The retractor system may be configured to retract not only checks and lips but also protect or shield other soft tissues such as the tongue or floor of the mouth. This may improve treatment access, enhance patient safety, and minimize unintended current paths.

The check retractor system may comprise one or more of the following retention mechanisms: (1) Clip, Hook, or Channel: A fixed or movable feature that engages with the electrode wire or terminal to maintain its position during treatment; (2) Keyed or Nested Interface: A geometry on the retractor that matches a corresponding shape on the electrode or wire terminal 222 (See FIG. 7), ensuring correct orientation and or preventing accidental detachment; (3) Adhesive-Backed Retention Clip: A disposable clip with adhesive backing that can be affixed to third-party check retractors, enabling retrofit of existing clinical devices; and/or (4) Magnetic or Friction-Fit Holders: Mechanisms that secure wires or terminals through non-permanent engagement.

In some embodiments, the retention structure is positioned such that it eliminates or redirects tension from the wire, allowing the electrode to adhere lightly to the gel and maintain stable contact with the teeth. The wire may pass through the clip and be secured by a flared stop or enlargement at its distal end.

In some embodiments, the attachment mechanism is configured to act as a mechanical fuse, releasing the wire or electrode from the retractor when subjected to a defined threshold of tension. This release force may be calibrated to remain below the force required to dislodge or remove the cheek retractor itself from the patient's mouth, thereby allowing safe decoupling of the treatment component without disturbing the overall intraoral setup.

In various embodiments, the cheek retractor system enables the following clinical advantages: (1) Strain Relief: Prevents wire tension from displacing the electrode or disrupting gel contact; (2) Stable Electrode Placement: Maintains consistent positioning of the electrode strip across one or both arches; (3) Improved Workflow: Allows hands-free treatment, reducing clinician burden and enhancing repeatability; (4) Modular Compatibility: Supports integration with various electrode designs and controller 100 (see FIG. 1) configurations.

In some embodiments, the retractor includes multiple clips 220 (see FIG. 7B) or retention sites, enabling simultaneous treatment of both arches or multiple treatment regions. In other embodiments, the system may be used with short or trimmed electrodes configured for single-tooth treatment.

In certain configurations, the cheek retractor may include one or more attachment points for a return electrode, such as a stainless-steel lip hook or intraoral contact. This integrated configuration may improve patient comfort, reduce external wiring, and simplify device setup. The return electrode may share a mechanical interface with the treatment electrode or be retained independently.

Example configurations can include: (1) A commercially available check retractor with two integrated clips molded from flexible plastic, each holding the lead wire for an upper and lower arch electrode; (2) A retractor-mounted channel that accepts a keyed terminal from a laminated electrode strip, providing strain relief and positional indexing; (3) A disposable retention clip with adhesive backing affixed to a third-party retractor to hold a single-tooth electrode wire; and/or (4) A retractor system incorporating a conductive lip hook as a built-in return electrode, electrically insulated from the primary structure.

In various embodiments, the check retractor system may incorporate any of the following variations permanent or removable retention clips; one-piece or modular construction; reusable or single-use materials; integration with gel delivery, light-curing, or suction components; color-coded or indexed positions for proper wire placement; and/or positionally adjustable clips for different arch sizes.

In various embodiments, the cheek retractor system described herein is designed to interface directly with: the electrode system described in this application, providing physical and mechanical support; the treatment platform described in this application, forming part of the integrated clinical workflow; optional barrier layers, which may be applied around or under the retractor clips; and/or the controller described in this application, via electrical leads routed through retractor-mounted clips. The retractor may also support standalone treatment configurations or be used in non-EKF applications that require secure intraoral placement of treatment components.

### Gingival and Oral Insulation Techniques for Electrokinetic Treatment

The electrokinetic delivery of agents into or through teeth requires the application of electric potential across a formulation in contact with the enamel or other mineralized surface. However, the oral environment includes numerous highly conductive soft tissues—such as gingiva, checks, and tongue—that, if contacted directly by the gel or electrode 110 (see FIG. 2), can allow unintended current pathways, resulting in discomfort, short circuiting, or ineffective treatment.

Furthermore, many patients have non-homogeneous dental anatomy, including crowns, fillings, missing teeth, or areas of decay that require targeting or exclusion during treatment. A uniform approach to electrical isolation is needed to (1) maintain consistent field strength through the enamel, (2) protect soft tissues, and (3) allow selective targeting of delivery sites.

The disclosed system includes a variety of electrically insulating barrier 130 (see FIG. 9) materials and methods for their application in order to: prevent unintended current flow through soft tissue or metal restorations; improve targeting of EKF treatment to desired sites (e.g., active lesions, decalcified areas); enable longer treatment durations by stabilizing the electrode-gel 112 (see FIG. 2) interface; support patient safety and comfort through reliable electrical isolation; and/or the insulating barrier may be placed between the formulation and surrounding oral structures or used to completely encapsulate the treatment site or components.

In various embodiments, suitable electrically insulating barrier material 130 (see FIG. 9) include, but are not limited to: photocurable polymer resins, including materials used in gingival barrier gels for professional whitening; silicone or rubber-based dental dams; viscous or semi-solid pastes, such as petroleum jelly or wax-based compounds; flowable materials that cure in ambient conditions (e.g., air-curable elastomers); and/or barriers that polymerize upon exposure to heat, friction, chemical initiators, or applied voltage. Example barrier materials can include: non-conductive or minimally conductive (e.g., higher resistivity than enamel); biocompatible and safe for intraoral use; easily applied and removed in a clinical setting; stable under treatment conditions (e.g., in the presence of peroxide or elevated voltage).

The barrier material may be applied by one or more of the following techniques: syringe extrusion and manual shaping; paint-on or brush-on delivery; wiping or pressing into place using a disposable applicator; air-drying, blow-curing, or light-curing in situ. In some embodiments, the gingival area is pretreated prior to application of the barrier material, such as: drying the surface using suction or air; and/or applying an astringent to shrink tissue and enhance adhesion; cleaning the margin to improve seal formation and reduce leakage.

In certain embodiments, one or more preparatory materials may be applied to reduce salivary interference, including absorbent pads, suction devices, or tissue-drying agents (e.g., astringents or desiccants) to enhance barrier or electrode adhesion.

In certain embodiments, the insulating barrier is used to support or stabilize the electrode itself. For example: the barrier may act as a wedge or cushion to hold the electrode in place during treatment; the barrier may be applied over the electrode, partially or fully encapsulating it to prevent movement; and/or the electrode may be embedded in the barrier material during curing to create a stable treatment interface. This can allow for extended-duration treatments, reduced sensitivity to patient movement, and potential treatment outside of direct supervision.

In various embodiments, the barrier material may be used not only to insulate non-target tissues, but also to mask portions of a tooth surface. In such embodiments: areas of intact or healthy enamel may be covered to exclude them from treatment; areas of carious demineralization, white spot lesions, or restorative margins may be selectively left exposed; and/or teeth not intended for treatment (e.g., those with crowns, veneers, or metal restorations) may be isolated entirely. This can enable site-specific electrokinetic treatment even in heterogeneous or partially restored dentitions.

In some embodiments, elastomeric or deformable insulating elements may be positioned between the electrode and surrounding tissue to improve comfort, shield soft tissue, or provide electrical isolation. Conductive adhesives or shielded return paths 116 (see FIG. 1) may be employed to maintain consistent circuit performance while minimizing unintended tissue contact.

In some embodiments, the barrier system enables non-adjacent teeth to be treated simultaneously using a single electrode, by masking intervening structures. For example: tooth 1 and tooth 3 may be treated while tooth 2 (containing a metal crown) is isolated by barrier material; and/or barrier material may separate independent gel pools while maintaining electrical contact via a spanning electrode strip. This can enhance treatment versatility and enables patient-specific configurations.

Example configurations can include: (1) A photocurable resin barrier applied along the gingival margin of the upper anterior teeth, isolating the enamel from soft tissue and enabling full-arch treatment with a surface-adhered electrode; (2) A viscous petroleum-based barrier molded to isolate a single molar for targeted remineralization; (3) A cured silicone ring applied around a white spot lesion on the buccal surface, insulating surrounding enamel; and/or (4) A full encapsulation of electrode and gel under a cured barrier layer to permit walking treatment post-appointment.

In various embodiments, the barrier system may include: multiple barrier types in the same treatment (e.g., photopolymer plus wax); electrically insulating adhesives with added viscosity for gel containment; colorants or indicators to assist visualization during placement; pre-formed or pre-sized barrier templates for rapid application; and/or preloaded barriers with integrated gel and/or electrodes.

In various embodiments, the barrier system interfaces with: the electrode system described in this application, by stabilizing or selectively isolating its contact with the oral environment; the overall platform, ensuring treatment specificity and minimizing current leakage; the cheek retractor 120 (see FIG. 7) system described in this application, which may assist in maintaining barrier placement during treatment; and/or the controller 100 (see FIG. 1) system described in this application, which relies on proper insulation to enable accurate fault detection and safe operation. The use of appropriate insulating barriers can enhance the precision, safety, and adaptability of electrokinetic dental treatments.

### Voltage Ramping and Current Control for Safe Electrokinetic Treatment

In electrokinetic dental treatment, electric current is applied to a treatment composition 112 (see FIG. 2) in contact with tooth surfaces to facilitate directional transport of therapeutic or cosmetic agents. While effective, even small unintended surges in applied voltage or current-especially at treatment initiation—can cause transient pain, patient discomfort, or injury if improperly controlled. Enamel is a highly resistive structure, and even microamp-level currents can cause sensation if abrupt transitions occur or if current flows through soft tissue rather than enamel.

To ensure that treatment remains painless and imperceptible under varying clinical conditions, it is necessary to precisely manage: the initial application of voltage (to avoid perception); the amount of current delivered (to avoid soft tissue stimulation or short circuiting); and/or the response to changes in electrical conditions during treatment (e.g., patient movement, dislodged electrode 110 (see FIG. 2), moisture ingress). The invention provides a controller 100 (see FIG. 1) system configured to perform real-time monitoring and adaptive control of voltage and current to ensure safe and effective EKF operation across a wide range of patient presentations.

The control system is operably coupled to the power source 102 and the electrode output port 104 and comprises logic and hardware elements configured to: (1) perform a pre-treatment circuit integrity check; (2) initiate a voltage ramp 302 (see FIG. 10) to a defined target voltage; (3) continuously monitor current during treatment; (4) apply current limiting based on software logic, analog circuitry, or both; (5) detect and respond to fault conditions, such as short circuits or open circuits; and/or (6) optionally alert the user to the system status or fault condition.

The control system may be implemented in hardware, firmware, software, or a combination thereof. In some embodiments, current limiting is achieved using one or more analog circuit elements such as resistors, diodes, or current-limiting devices. In other embodiments, or in conjunction with such hardware, software-based logic dynamically adjusts output voltage or duty cycle to maintain current within predefined safety thresholds 404 (see FIG. 11).

Multi-stage control logic may be used to enforce safety thresholds. For example, impedance checks, user confirmation, or physical interlocks may be required before initiating voltage delivery. The system may automatically disable output if electrical anomalies are detected.

In various embodiments, the power source may comprise a battery, wall-connected AC power supply, capacitive discharge unit, or other electrical source suitable for delivering a regulated voltage. The system may include onboard or external converters to accommodate different input types and voltages.

In some embodiments, the system is configured to ramp the applied voltage until a predefined current threshold is achieved, at which point voltage delivery is paused, modulated, or held constant. This approach allows for current-based dosing or comfort optimization based on measured patient impedance.

To minimize patient discomfort, the controller applies voltage using a gradual ramp 302 (see FIG. 10), rather than a step function 300 (see FIG. 10). In some embodiments: (1) the ramp may be linear, exponential, or defined by a user-adjustable profile; (2) ramp rates up to approximately 10 volts per second have been found to be imperceptible at operating voltages up to 30 V; and/or (3) the ramp may be executed continuously or in discrete steps. In various embodiments, the ramp initiates only after confirmation that the electrode circuit is intact and that the treatment site is properly configured.

The applied voltage or current may follow a range of time-varying profiles including exponential rise, stepwise ramps, decaying pulses, or alternating polarity signals. In certain embodiments, high-voltage pulses or polarity reversal may be employed to improve transport efficiency or penetration.

The system may be configured to operate using DC, pulsed DC, or modulated AC signals. Waveform characteristics such as duty cycle, frequency, or amplitude may be selected to optimize comfort, treatment efficacy, or safety.

Prior to initiating full treatment voltage, the system may: (1) apply a low test voltage to the circuit; (2) measure the resulting current or impedance 118 (see FIG. 1); and/or (3) determine whether the gel-electrode interface and return path 116 (see FIG. 1) form a complete circuit. In some embodiments, the system uses this measurement to: (1) confirm that the electrodes are correctly positioned; (2) establish a baseline impedance value for reference 402 (see FIG. 11); and/or (3) delay treatment until the circuit meets predefined thresholds 404. This pre-check can improve both safety and treatment reliability.

In various embodiments, throughout treatment, the system monitors the current delivered to the treatment site. The system can include one or more of the following current-limiting mechanisms to keep the applied current below a desired threshold 304 (See FIG. 10) with the goal of ensuring safety and no perception by the patient: (1) current-limiting diode or resistor to cap physical current; (2) digital current sensing circuit to measure live current flow; (3) comparator logic to determine if a threshold is exceeded; and/or (4) interrupt or fuse system to halt voltage application if limits are breached.

In some embodiments: the current limit is user-adjustable, based on treatment area or patient preference; the system supports asymmetric limits for different arches or electrode zones; the device may ramp down voltage dynamically to stay within limits; and/or the current-limiting threshold is configured to allow effective electrokinetic transport without reaching the human perception threshold, for example, below 100 μA per tooth.

In certain embodiments, one or more of these safety features may be integrated directly into the disposable electrode or peripheral device. For example, a current-limiting resistor, diode, fuse, or comparator circuit may be embedded within the electrode lead, terminal housing, or connector, enabling passive or semi-intelligent protection even when the controller is a low-cost, naïve power source 102 (e.g., a constant-voltage battery pack). This approach may be advantageous in outpatient, at-home, or mobile treatments where minimal controller complexity is desired. Such disposable-integrated safety features may act independently or in conjunction with upstream digital controls in the main device.

In various embodiments, if the current exceeds or falls below expected ranges, the system identifies this as a potential fault condition, including: short circuiting (e.g., gel contacting gingiva or lips); open circuit (e.g., detached electrode or missing return path); and/or unstable impedance due to patient movement or saliva contamination. In various embodiments, upon fault detection, the controller may: immediately terminate voltage application; illuminate an indicator or issue a digital alert to notify the user; log the fault event for traceability; and/or prompt the user to inspect and reposition components.

In some embodiments, the controller supports automated recovery behaviors, such as: applying a test voltage following a fault to re-evaluate impedance; re-applying the voltage ramp if conditions are restored and safe 412; storing the initial impedance value, and using it to guide safe reactivation; and/or adjusting voltage delivery dynamically to achieve sub-threshold current during post-fault treatment. This capability can enable real-time adaptability and reduces unnecessary treatment interruptions due to transient issues (e.g., brief contact with tongue or saliva).

In various embodiments, the system may include one or more visual or electronic indicators showing: treatment status (e.g., ramping, active, completed); fault status (e.g., current exceeded 304 (see FIG. 10), electrode disconnected); arch-specific current flow (e.g., left vs. right, top vs. bottom); and/or power levels or operational readiness. Indicators may be implemented using LEDs, displays, acoustic signals, or wireless communication protocols.

In various embodiments, the device control system operates according to a defined sequence of monitoring and response steps to ensure safe, effective electrokinetic treatment under varying intraoral conditions. The updated logic flow is illustrated in FIG. 11 and includes both preventive checks and adaptive responses.

The following is a logic flow. Upon powering on (400 Device On), the system initiates a pre-treatment current or impedance check (402 Current Check) to determine whether a complete and conductive circuit exists between the active electrode and the return path. This check may involve applying a low-test voltage and measuring resulting current to assess system continuity and readiness. If the measured current falls within an acceptable operational range (404 Acceptable Current?), the system proceeds to ramp voltage to the desired treatment level (406 Apply Ramped Voltage), using a time-based control profile (e.g., linear or exponential ramping). This gradual increase is designed to avoid patient perception and ensure stable initiation of electrokinetic flow.

While treatment is active, the controller continuously monitors delivered current (408 Monitor Current) to detect anomalies. If no fault is detected (410 Fault Detected?=No), treatment continues without interruption (416 Continue Treatment). If a fault is detected—such as a current spike (e.g., short circuit) or current drop (e.g., open circuit)—the system evaluates whether retry behavior is allowed (412 Retry Permitted?).

If retries are disallowed due to prior failures or system settings, the controller enters an error lockout state (416 Error Lockout), requiring manual intervention. If retry is permitted, the system enters a delay or cooldown phase (414 Wait and Retry) to allow stabilization (e.g., patient re-positioning, gel resettling). After the wait period, the voltage ramp is re-initiated (406 Apply Ramped Voltage) and treatment resumes if circuit conditions are acceptable.

This architecture ensures robust fault recovery and patient safety while minimizing false positives and unnecessary treatment interruptions. Each stage in the control sequence includes real-time sensing and can optionally trigger visual or electronic indicators to notify clinical staff of system status.

In some embodiments, the current check 402 is configured to operate across multiple independent electrodes or channels, such as in a dual-arch treatment configuration. The system may assess circuit continuity and impedance separately for each connected electrode—e.g., one spanning the upper arch and another spanning the lower arch—and may require that all active electrodes establish complete circuits before initiating treatment. For example, in a configuration where both arches are treated simultaneously, the system may prevent voltage ramping 406 unless both the maxillary and mandibular electrode paths pass the current check threshold. The current check may likewise monitor any number of active channels, input terminals, or treatment zones, and may be used to enforce channel-specific ramping, current limiting, or fault isolation logic. This approach enables flexible configurations while ensuring treatment only proceeds under safe and verified electrical conditions.

In certain embodiments, the controller interfaces with a treatment timer or dosage control subsystem configured to regulate energy delivery based on time, current, or both. The timer may operate in a constant-time mode, where a defined voltage or ramped voltage is applied for a fixed duration (e.g., 60 seconds), or in a current-limited mode, where treatment continues until a target cumulative current (e.g., total microamp-seconds) has been delivered. These modes may be selected based on clinical indication, patient sensitivity, or treatment formulation. For example, the system may be configured to terminate treatment once a total of 300 μA has passed through the circuit, or to maintain delivery for a predetermined 90-second window. In some implementations, both time and current thresholds may be applied simultaneously, with treatment ceasing upon whichever condition is met first. This enables flexible and safe dosing strategies tailored to diverse patient and material profiles.

In some embodiments, the treatment timer interfaces with the fault detection and retry system to determine whether continued attempts at voltage reapplication are appropriate based on the remaining treatment time and likelihood of successful recovery. For instance, if a fault is detected late in the treatment cycle—such that insufficient time remains to allow repositioning or re-initialization of the electrode—the system may opt to withhold reapplication of voltage, but continue displaying that the treatment session is active until the timer expires. This prevents misleading interruption indicators while acknowledging that no further current will be delivered. In other cases, the system may detect an unrecoverable fault (e.g., a persistent short circuit due to a cracked gingival barrier 130) and intelligently suppress voltage output while maintaining session status, enabling the clinician to complete other steps (e.g., finishing chairside tasks) without triggering alarms or requiring unnecessary teardown. This behavior supports more graceful error handling, reduces false alarms, and accommodates real-world treatment dynamics where some faults may not be correctable mid-session.

In some embodiments, the treatment system includes a multi-phase timer or programmable sequencing controller configured to guide the user through a series of treatment steps-some of which may involve active current delivery, while others may not. For example, a treatment protocol may begin with a preparation phase (e.g., a 3-minute countdown to allow for ethanol swabbing and dehydration of the tooth), followed by a current-enabled infiltration phase (e.g., 5 minutes of electrokinetic treatment), and conclude with a finishing phase (e.g., 2 minutes for applying and curing a protective or adhesive layer, without further current). These phases may be defined by the clinician or preloaded as presets associated with different treatment types (e.g., whitening, remineralization, adhesion). The timer may continue to run across all phases—either as a single countdown or as distinct steps—while selectively enabling or disabling current delivery depending on the protocol requirements. This functionality enables the system to act as a stepwise clinical guide, improving procedural consistency, reducing reliance on external timers or verbal instructions, and ensuring that each step is delivered for the appropriate duration with visual or electronic prompts provided throughout.

Example configurations can include: (1) a portable device with a digital controller that ramps voltage from 0 to 30 V over 5 seconds, then maintains steady voltage while limiting current below 150 μA; (2) a system that detects short circuiting within 50 ms, halts treatment, and prompts the clinician to reposition the electrode; (3) a device that records impedance at initial placement and uses that value to determine whether re-ramping is safe after fault clearance; and/or (4) a user-configurable controller that allows selection of ramp rate, current threshold, and alert mode.

The control system may be configured to support: multiple modes (e.g., whitening, remineralization, desensitization) with different ramp and current profiles; manual override of voltage thresholds; integration with mobile applications or external data logging; treatment profiles customized for different anatomical regions; and/or automatic selection or recommendation of treatment mode based on impedance values measured at the treatment site. In some embodiments, the system detects electrical characteristics of the target area—such as surface conductivity, hydration level, or tissue impedance—and uses these values to select or suggest a mode appropriate for enamel, dentin, gingiva, or mucosal tissue. This feature may operate independently or in conjunction with preset user preferences or clinician guidance.

The controller system described herein is interoperable with: the electrode system described in this application, providing precise control over delivered current; the insulation and barrier 130 (see FIG. 9) system, which can minimize unintentional current paths; the cheek retractor 120 (see FIG. 7), which can assist in electrode and wire placement for optimal contact and current control; the overall treatment platform described in this application, ensuring safe and effective operation across varied clinical workflows. Together, these elements can form a comprehensive system for electrokinetic dental treatment with safety, adaptability, and minimal patient perception.

### Compatible Treatment Formulations

Electrokinetic delivery relies on the presence of a conductive medium—typically in the form of a gel 112 (see FIG. 2), paste, film, varnish, suspension, or solution—positioned in contact with the treatment site. The disclosed systems are designed to operate with a broad range of such formulations, including commercially available or custom-prepared dental compositions. While electric field strength and direction are governed by the controller and electrode configuration, the conductive properties, viscosity, and electrochemical behavior of the formulation can influence treatment efficacy.

The systems disclosed in this application are compatible with diverse treatment formulations including, as some examples: conductive, ionic, polar, or otherwise field-responsive species. These formulations may be intended for: whitening or tooth shade modification; remineralization or lesion reversal; hypersensitivity reduction; antimicrobial or antibiotic delivery; anti-inflammatory or biologic treatment; diagnostic tracer or sampling applications; polymer, resin, or adhesion-related enhancements. The system does not rely on any specific formulation or agent class, and no single formulation is required for successful operation of the electrokinetic treatment platform.

In some embodiments, the gel or treatment composition 112 (see FIG. 2) may: contain active agents in ionic, polar, or charged molecular forms; exhibit a conductivity suitable for generating a stable electric field (e.g., via inclusion of buffer salts, conductive ions, or electrolytes); be thickened with materials such as fumed silica, cellulose derivatives, carbomers, polyacrylic acid, PEG, or hydrogels to improve retention; be formulated as low-viscosity liquids, self-supporting gels, sprayable emulsions, or injectable pastes, depending on the clinical application; include pH-modifying, chelating, or stabilizing agents to maintain chemical stability during direct current application; be retained in contact with the tooth using a barrier material, retractor-based support, or electrode surface tension; deliver single or multiple therapeutic agents, or serve as a vehicle for both treatment and sampling. The system does not rely on any specific formulation characteristics, and may be used with a broad range of substances and gel types, including off-the-shelf dental materials.

Insert Nothing in this specification should be interpreted as limiting the formulation to specific ingredients, compositions, physical forms, or rheological properties. The described system may be used with existing commercial dental materials or with novel formulations engineered for enhanced electrokinetic compatibility.

Custom or optimized compositions—such as those developed for superior whitening efficacy, enhanced delivery speed, selective tissue targeting, or reduced irritation—are explicitly preserved for future filings. This includes formulations that may use novel combinations, concentrations, excipients, stabilizers, or delivery methods beyond what is described here.

In various embodiments, the disclosed formulations serve as the medium for electrokinetic delivery and interface with: the electrode system described in this application, which delivers voltage across the composition to generate field-driven transport; the controller and power supply, which can regulate voltage ramping, duration, and current limits; the barrier and retractor systems, which can help retain the formulation and isolate non-target tissues. Formulations may be applied using standard dental techniques (e.g., brush, syringe, tray, or sponge), or through integrated application systems that combine agent delivery with electrode placement in a single workflow.

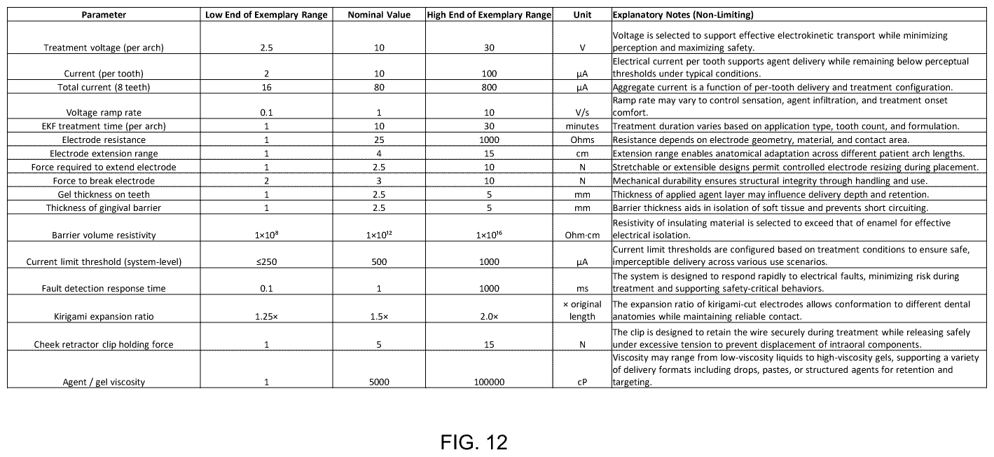

Each numerical value presented herein is contemplated to represent a minimum value or a maximum value in a range for a corresponding parameter. Accordingly, when added to the claims, the numerical value provides express support for claiming the range, which may lie above or below the numerical value, in accordance with the teachings herein. Every value between the minimum value and the maximum value within each numerical range presented herein (including in the chart shown in FIG. 12), is contemplated and expressly supported herein, subject to the number of significant digits expressed in each particular range. FIG. 12 also provides express support for the ranges between minimal and nominal, nominal and maximum, and minimum and maximum for each parameter listed.

Having described herein illustrative embodiments of the present invention, persons of ordinary skill in the art will appreciate various other features and advantages of the invention apart from those specifically described above. It should therefore be understood that the foregoing is only illustrative of the principles of the invention, and that various modifications and additions can be made by those skilled in the art without departing from the spirit and scope of the invention. Accordingly, the appended claims shall not be limited by the particular features that have been shown and described but shall be construed also to cover any obvious modifications and equivalents thereof.

1. A device for applying an electric potential to a treatment site, the device comprising:
a power source adapted to provide energy for delivery to the treatment electrode;
a treatment electrode adapted for placement proximate the treatment site;
a reference electrode; and
a control system adapted to perform operations comprising:
confirming that a complete circuit is established between the treatment electrode and the reference electrode;
upon confirmation of the complete circuit, initiating a voltage ramp from an initial voltage to a target treatment voltage over a predetermined time interval;
monitoring current delivered to the treatment electrode during the voltage ramp and a treatment period; and
if the monitored current meets a predetermined condition, terminating voltage delivery.

2. The device of claim 1, wherein the treatment site comprises a dental treatment site.

3. The device of claim 2, wherein the treatment electrode is adapted to contact a conductive agent applied to the treatment site.

4. The device of claim 3, wherein the conductive agent comprises a gel-formulated agent.

5. The device of claim 3, wherein the conductive agent is selected from the group consisting of a tooth whitening or shade-modifying agent, a remineralizing or desensitizing agent, and an antimicrobial or antibiotic agent.

6. The device of claim 5, wherein the remineralizing or desensitizing agent is selected from the group consisting of fluoride salts, zinc salts, calcium phosphate compounds, and potassium nitrate.

7. The device of claim 5, wherein the antimicrobial or antibiotic agent is selected from the group consisting of chlorhexidine, triclosan, and a tetracycline-class compound.

8. The device of claim 1, further comprising an electrically insulating barrier adapted to be applied to a region adjacent the treatment site.

9. The device of claim 8, wherein the region adjacent the treatment site comprises gingival tissue.

10. The device of claim 8, wherein the electrically insulating barrier is light-curable and adapted to conform to the region adjacent the treatment site.

11. The device of claim 1, wherein confirming that the complete circuit is established comprises applying a low test voltage and detecting that a resulting current exceeds a test threshold value.

12. The device of claim 1, further comprising a current-limiting element adapted to limit an amount of current delivered to the treatment electrode.

13. The device of claim 12, wherein the current-limiting element is selected from the group consisting of: a fixed resistor, a fuse, and a diode.

14. The device of claim 1, wherein the device is adapted to interface only with components including an encoded connector, thereby restricting use to approved treatment conditions.

15. The device of claim 1, wherein the control system operations further comprise generating an alert indicating a treatment status or fault condition.

16. An electrode for use in delivering a treatment composition to a treatment site, the electrode comprising:
a flexible and electrically conductive body, wherein the conductive body is customizable in size such that it can be physically adapted to conform to a particular treatment site; and
an electrical connection terminal adapted to couple the electrode to a power source,
wherein the electrode is adapted to deliver electric current through the treatment composition to facilitate transport of active agents into the treatment site via at least one of electrokinetics and iontophoresis.

17. The electrode of claim 16, wherein the treatment site comprises a dental treatment site.

18. The electrode of claim 17, wherein the conductive body comprises a size sufficient to span across multiple teeth.

19. The electrode of claim 16, wherein the conductive body comprises a laminated structure comprising a plastic backing layer, an adhesive layer, and a conductive metallic foil.

20. The electrode of claim 16, wherein the conductive body is adapted to fit to the treatment site via at least one of structural perforations, expansion joints, or patterned slits.

21. The electrode of claim 16, wherein the electrode is further adapted to be mechanically secured to a stabilizing structure during treatment.

22. The electrode of claim 21, wherein the stabilizing structure comprises a cheek retractor.

23. The electrode of claim 16, wherein the electrode is adapted to deliver electric current through the treatment composition to facilitate transport of active agents into the treatment site via at least one of electrophoresis and electroosmosis.

24. A retractor comprising:
a retractor element adapted to retract soft tissue from a treatment site; and
at least one retention structure operably connected to the retractor element and adapted to removably secure a treatment component proximate the treatment site.

25. The retractor of claim 24, wherein the treatment site comprises a dental treatment site.

26. The retractor of claim 25, wherein the soft tissue comprises a cheek tissue.

27. The retractor of claim 24, wherein the treatment component is adapted to interact with a substance applied to the treatment site.

28. The retractor of claim 24, wherein the treatment component at least partially enters the oral cavity.

29. The retractor of claim 28, wherein the treatment component directly contacts an intraoral tissue or substance.

30. The retractor of claim 24, wherein the retention structure comprises at least one of a clip, a hook, a loop, or a channel.
